(19) United States
(12) Patent Application Publication
(10) Pub. No.: US 2022/0001553 A1
(43) Pub. Date: Jan. 6, 2022

(54) MAGNETIC LOCKING UTILITY KNIFE

(71) Applicants: Dilantha B Ellegala, Lynchburg, VA (US); Robert Trimarche, Lynchburg, VA (US); Calvin Dudley, Lynchburg, VA (US)

(72) Inventors: Dilantha B Ellegala, Lynchburg, VA (US); Robert Trimarche, Lynchburg, VA (US); Calvin Dudley, Lynchburg, VA (US)

(21) Appl. No.: 16/921,441

(22) Filed: Jul. 6, 2020

(51) Int. Cl. B26B 5/00

(52) U.S. Cl. CPC B26B 5/003; B26B 11/00; B26B 5/008

(57) ABSTRACT

A utility tool device including a handle with a tool recess and a lever arm recess; a rotatable lever arm with a magnet that is configured to fit in the lever arm recess; and a rotatable lever hinge attached to the lever arm and configured to attach to a tool; wherein the tool recess is configured to have a tool fit in the tool recess; and wherein the magnet is configured to lock a tool in place when the lever arm is in the lever arm recess.

### CROSS-REFERENCE TO RELATED APPLICATION

This is a Continuation of U.S. application Ser. No. 15/529,199 filed on May 24, 2017, which claims priority from U.S. Provisional Application No. 62/083,497 filed Nov. 24, 2014, the disclosure of which is incorporated herein by reference.

### BACKGROUND

### 1. Field

Exemplary embodiments relate to utility knife, in particular, to a utility knife having a safer blade extending/retracting mechanism with a unique design for a magnetic locking handle.

### 2. Description of the Related Art and Background

Utility knives are commonly used in various settings for a myriad of purposes. There are currently multiple designs for extending and retracting the blade of these utility knifes. Locking mechanisms for maintaining exposed blades can be worn out overtime, exposing the user to potential hazardous injury due to the blade not maintaining its fixed position.

It is therefore an object of this invention to provide a safe utility knife with a retractable blade that prevents or at least minimizes the chance of operator injury with exposure/retraction of the blade surface.

It is still a further object of this invention to provide a unique sliding and pivoting locking mechanism for exposure and retraction of the blade.

It is yet a further object of this invention to provide a magnetic locking mechanism for the sliding/pivoting locking mechanism of the blade handle.

It is also an object of this invention that the unique design and use of the blade/handle locking mechanism be universal for multiple applications not listed herein.

Yet another object of this invention is the modification of the base design to allow for detachable blades.

Still yet another object of this invention is the modification of the base design to allow for multiple blade attachments for various needs.

### SUMMARY OF THE INVENTION

This invention relates to a utility knife with a retractable blade that extends and retracts out of a uniquely designed handle that has a unique sliding mechanism for exposing/retracting the blade with a magnetic locking feature. This will allow for quick and safe exposure of the blade when in use, without the need for the operator to expose themselves to the cutting edge of the blade. The handle will have a lever arm that can be extended from either side, which is attached to the blade to extend/retract the blade in/out of the handle base. Once the blade is fully exposed or retracted, the end of the handle will rotate 90 degrees back to the base, where it will magnetically be held and locked in place. Variations of the retractable blade design can be fashioned for various tools, including but not limited to: screwdrivers, rulers, lights, scissors, tweezers, writing utensils, etc. There are also several modifications that can allow for multiple blades from both ends of the handle as well as detachable blades.

Exemplary embodiments of the invention relate to a utility knife, in particular, to a utility knife having a safer blade extending/retracting mechanism with a unique design for a magnetic locking handle. This design will protect the user from inadvertent blade exposure when the blade is being exposed and/or retracted.

On both lateral sides of the handle, there is a recessed lever surface that can be pulled out from the base and rotated on a swiveling joint at a 90° angle. This lever surface is attached to the blade, which allows the blade to be extended in a parallel fashion from the handle base. The lever which is perpendicular from the handle base can then be rotated back to the base. There is a small magnet embedded into the recessed lever surface when rotated back into the handle base will maintain its locked position.

The blade can be substituted by various tools dependent on specific needs not listed herein. The recessed lever arms can be positioned at the proximal and distal end of the handle, as well as both sides, increasing the option of up to four blades.

Another embodiment of the current invention is the unique design element/mechanism of the lever arm hinge attachment to be embedded into the center spacer bar, via a recessed space to allow for rotation of the hinge and a uniquely designed detachable blade used in prior art for safe removal and attachment of said blade.

Exemplary embodiments of the current invention include a utility tool device, with a handle with a tool recess and a lever arm recess; a rotatable lever arm with a magnet configured to fit in the lever arm recess; and a rotatable lever hinge attached to the lever arm and configured to attach to a tool; wherein the tool recess is configured to have a tool fit in the tool recess; and wherein the magnet is configured to lock a tool in place when the lever arm is in the lever arm recess. In addition, the lever hinge may be rotatable about an axis perpendicular to the tool and the lever arm may be rotatable approximately 90 degrees about the lever hinge.

### BRIEF DESCRIPTION OF THEDRAWINGS

These and/or other aspects will become apparent and more readily appreciated from the following description of exemplary embodiments, taken in conjunction with the accompanying drawings of which:

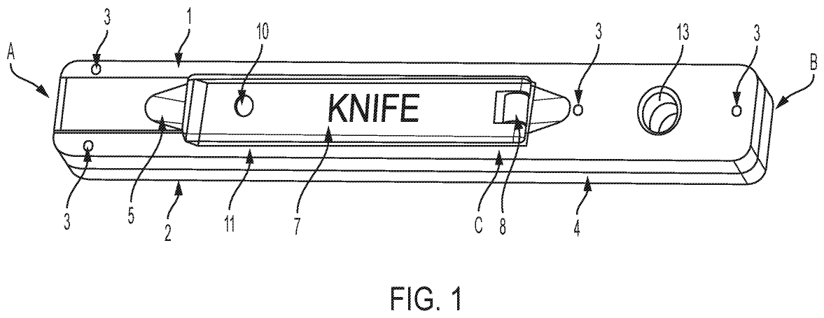

FIG. 1 is a side perspective view of an exemplary embodiment of the utility knife of the present invention.

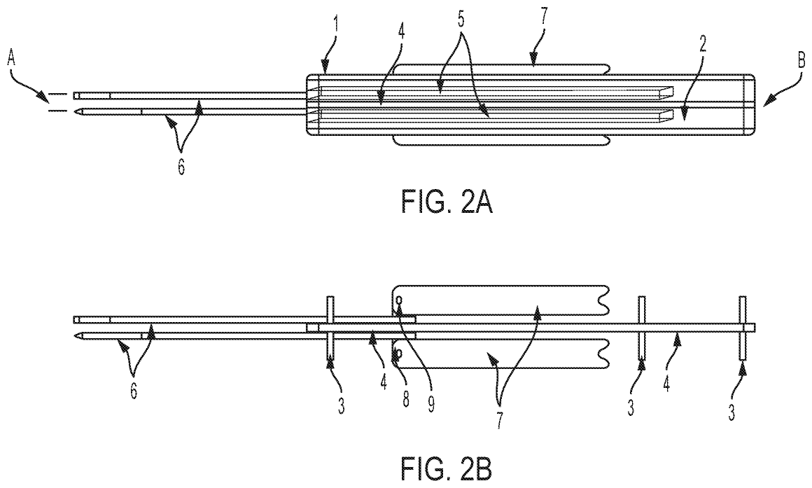

FIGS. 2A and 2B show top views of an exemplary embodiment of the utility knife of the present invention.

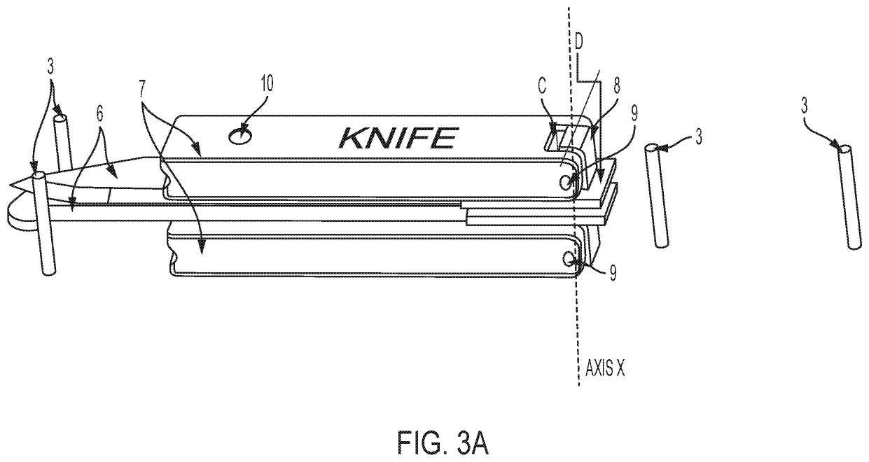

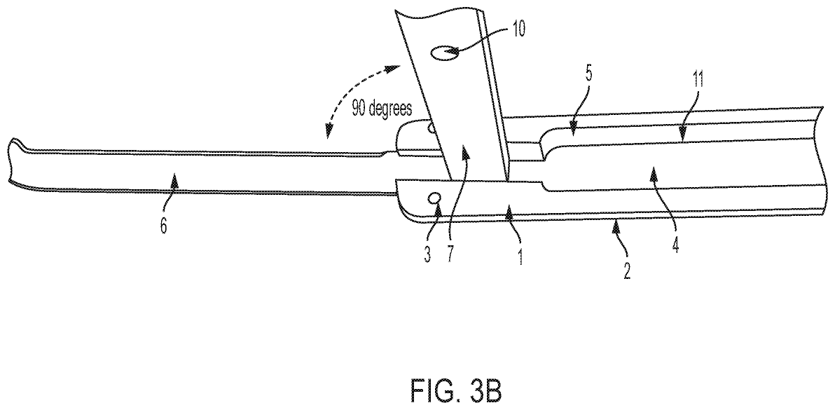

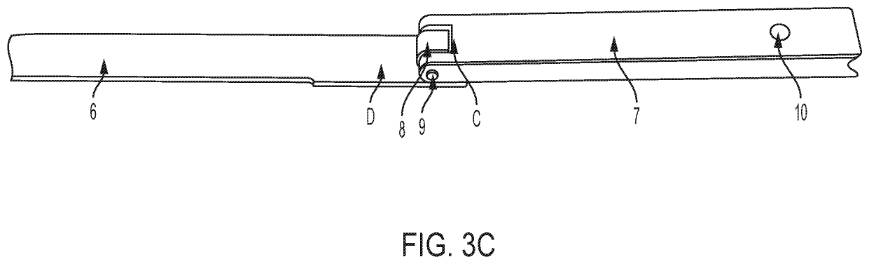

FIGS. 3A-3C show a partial side perspective views of an exemplary embodiment of the utility knife of the present invention.

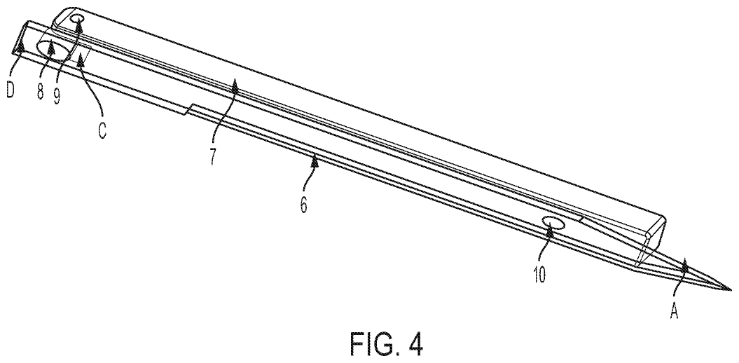

FIG. 4 demonstrates the closed lever arm with magnet keeping the blade in stored resting position.

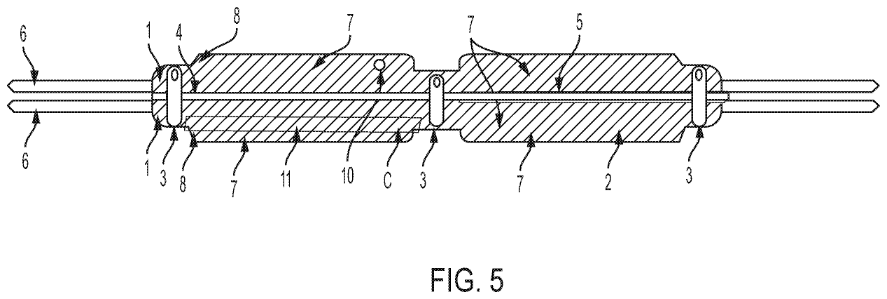

FIG. 5 shows the multi-level 4 blade construction.

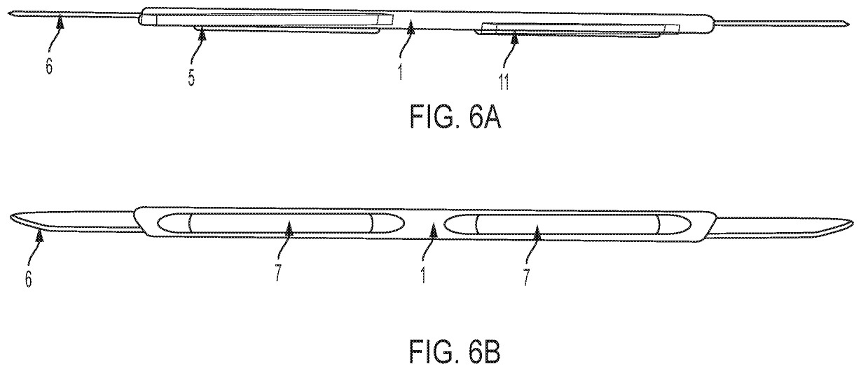

FIGS. 6A-6B show surgical blades attached to the knife.

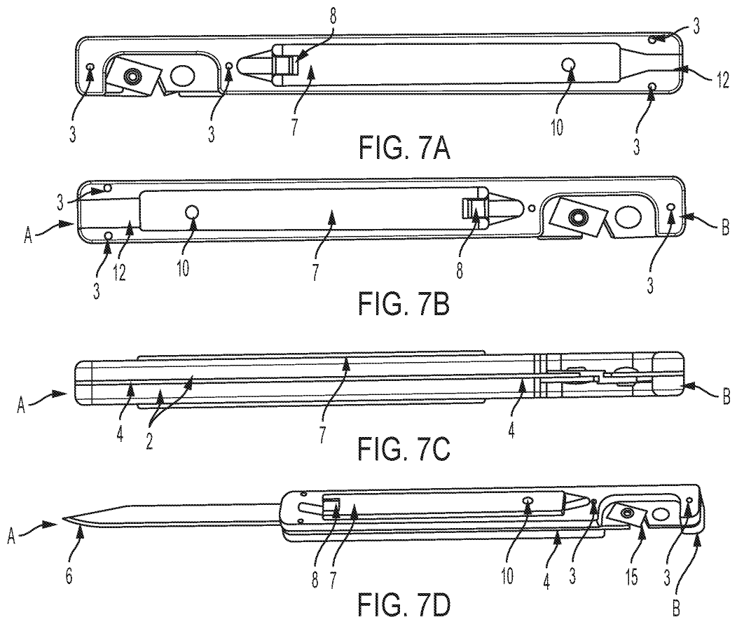

FIGS. 7A-7D show multi-views with the removable bladed knife handle, and the integrated knife sharpener.

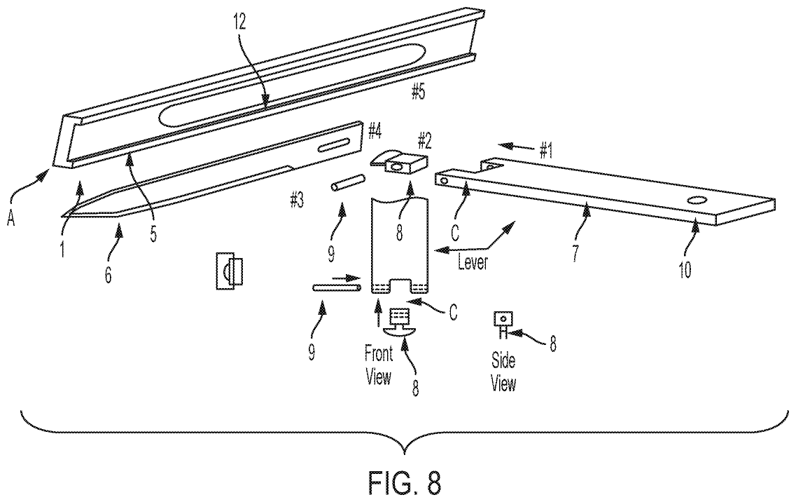

FIG.8 shows the knife in an exploded view, with the embodiment of the removable blade.

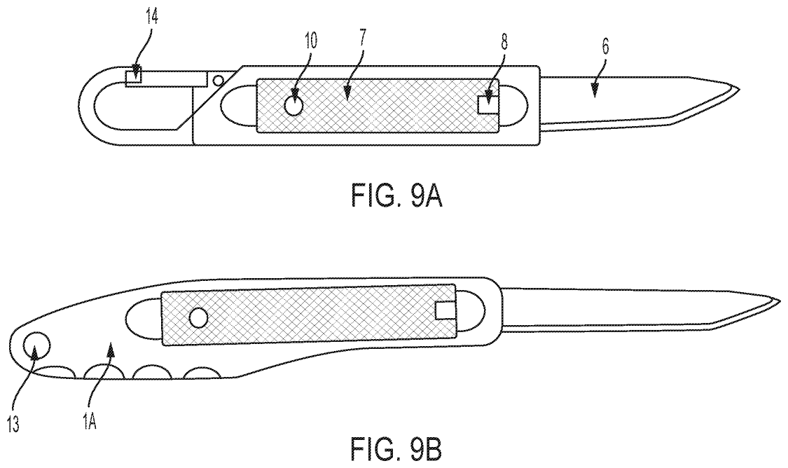

FIGS. 9A and 9B show modifications to the handle base, including a carabineer clip in FIG. 9A and an ergonomic handle with lanyard hole in FIG. 9B.

### DETAILED DESCRIPTION

Reference will now be made in detail to exemplary embodiments, examples of which are illustrated in the accompanying drawings, wherein like reference numerals refer to like elements throughout. However, known functions associated with the exemplary embodiments or detailed descriptions on the configuration and other matters which would unnecessarily obscure the present disclosure will be omitted.

Although exemplary embodiments of the disclosure have been shown and described, it would be appreciated by those skilled in the art that changes may be made in these exemplary embodiments without departing from the principles and spirit of the exemplary embodiments, the scope of which is defined in the claims and their equivalents. For example, while one use of the invention is in the medical field as a surgical scalpel, the same principles could be used to make devices for other medical and non-medical fields.

The embodiment of the current invention is shown generally in FIG. 1. While the embodiments shown below refer to blades, the invention is not limited to blades and could be used with other compatible tools. It includes handles 1, 2, pins 3, center spacer bar 4, internal blade track 5, blade lever arm 7, blade lever hinge 8, locking magnet 10 and a recess cut out 11 in the handle 1 (lever arm rest). The knife has a front/proximal end or nose A, rear/distal end or butt B, and distal end of the blade lever arm cut out C.

FIG. 2A shows a side view with the handles 1, 2 in place and internal blade tracts 5. FIG. 2B shows the handles removed to reveal the inner working of the lever arms/lever arm pivots and their relation to the blades 6 and center spacer 4. The figures show handles 1, 2, pins 3, center spacer bar 4, internal blade track 5, blades 6 (the pointed blade may be a skinning blade and the flat ended blade may be an eviscerating blade), blade lever arm 7, blade lever hinge 8, lever hinge pin 9 and locking magnet 10. The knife has a front/proximal end or nose A and rear/distal end or butt B. In this figure, the blades are shown in an open position.

In these and the other figures, what is shown is a handle made of two housing portions 1, 2 made of various materials. The handle portions are secured to one another by pins 3, which are affixed to a center spacer bar 4, to form the complete knife housing. The knife handles 1, 2 have a generally elongated profile that may be comfortably grasped by a user's hand with the front/proximal end or nose A of the knife extending from the user's hand. Toward the rear/distal end or butt B of the handle 1, 2, variations of the current invention will house different tools, including but not limited to: lanyard hole, blade sharpener, bottle opener, other blade options, etc.

The two separate handle portions 1, 2 will have a recessed channel or internal track 5, that extends the length of the handle, longitudinally and proximal to the spacer bar4, where the separate blades/tools 6 will rest.

The handle body 1, 2 will also have a lever arm recess cut out 11, so that the blade lever arms 7 will rest in that space. At the distal end of the blade lever arm 7 will be a cut out space C where the blade lever hinge 8 will rest. Inside the lever hinge 8 will be a space for the lever hinge pin 9 to secure the lever arm 7 to the lever hinge 8 that is affixed to the distal end of the blade body D (see FIG. 3A).

FIG. 3A-3C show the handle 1, 2 and center spacer 4 removed, showing the lever arm 7, lever hinge 8 and the relative location of how they rest inside the handles 1, 2 in the closed position in relation to the blades 6.

In FIG. 3B the proximal end of the lever arm 7 is rotated to 90 degrees about said lever hinge and then the lever hinge is rotated about an axis perpendicular to the blade (AXIS X), enabling the user to slide the blade 6 into the open position. Once the blade 6 is fully extended, the lever hinge 8 will be rotated about the axis perpendicular to the blade attaching the blade and then the lever arm 7 will rotate downward to its resting position. This is shown in FIG. 3C with lever arm/hinge/blade mechanism in a locked position.

FIG. 4 shows the closed lever arm 7 with magnet 10 keeping the blade 6 in stored resting position. The lever arm 7 will move freely within a 180 degree plane about the lever hinge 8. In the closed position, the lever hinge 8 rests in the distal end of the handle rest recessed cut out as shown in FIG. 3A. The blade is locked in place by the lever arm 7 resting recessed in the handle rest and by a locking magnet 10 embedded in the proximal end of the lever arm 7. The magnet 10 holds to the metal in the blade and the center spacer.

FIG. 5 shows a multi-level four blade construction.

FIGS. 6A-6B show two surgical blades 6 attached to a two-sided knife.

FIGS. 7A-7C show multi-views with the removable bladed knife handle, and an integrated knife sharpener 15. It also shows a recessed cut out 12 in the handle for a removable blade.

FIG. 8 shows the knife in an exploded view, with the embodiment of the removable blade. This is accomplished by the entire lever hinge body being able to rotate 90 degrees along a perpendicular axis to the 180 degree lever hinge, locking the blade to the hinge and lever arm. These three components are locked to the handle via a blade recess channel 12 in the handle base, allowing the longitudinal movement of the blade 6. This is inserted into the handle and blade moves parallel to the handle base, as noted in preceding figures.

FIGS. 9A-9B show modifications to the handle base, including a carabineer clip 14 in FIG. 9A and an ergonomic handle 1A with lanyard hole 13 in FIG. 9B.

Features of embodiments of the invention may include any of the following:

1. a handle base with recess surfaces on both sides that will house lever arms.;

2. the recessed lever arms described above would be attached to a blade or other tool/device, so that the free end of the lever arm can be extended out of and parallel to the handle base in a longitudinal fashion.

3. the recessed lever arms of the handle could lock into a fixed position parallel and in-line with the handle base by way of a magnet and seating in the recessed space.

4. the recessed lever arms could be movable to 180° at a swiveling joint.

5. the lever arms could have a cut out designed to fit the hinge via a hinge pin, allowing for rotation in the center axis of the hinge pin.

6. the handle could have a longitudinal channel, allowing for movement of the blade by way of perpendicular force exerted along it at the hinge.

7. the variations of the current invention could include a double sided four blade variation (FIG. 5).

8. the variations of the current invention could include a surgical scalpel modification, (see FIG. 6A-6B) with two separate blade sides that can house different types of surgical blades. This can also be made of high grade plastic for single use in operating rooms and medical practices, as well as reusable handle base for use with replaceable blades.

9. the variations of the current invention include a removable blade by way of a blade lever arm hinge that is seated in a recess of the handle spacer and rotatable, to allow for a uniquely designed blade to be attached/removed (see FIGS. 7A-7D and 8). This is accomplished by the entire lever hinge body being able to rotate 90 degrees along a perpendicular axis to the 180 degree lever hinge, locking the blade to the hinge and lever arm. These three components are locked to the handle via a secondary recessed channel in the handle base, allowing the longitudinal movement of the blade. This is inserted into the handle and blade moves parallel to the handle base.

10. the variations of the current invention include a uniquely designed blade sharpener 15, made of high grade steel, ceramic or other commonly used hard surfaces, designed for sharpening blades; that is affixed to the distal end of the handle.

11. the variations of the current invention include fixation points for carrying the invention on the distal end of the knife, examples are shown in FIG. 1 with lanyard hole, in FIGS. 9A with a modified distal handle base with a carabineer type clip and in FIG. 9B with an oversized handle ergonomically designed for the hand for use in hunting and cleaning game animals.

Although exemplary embodiments of the disclosure have been shown and described, it would be appreciated by those skilled in the art that changes may be made in these exemplary embodiments without departing from the principles and spirit of the exemplary embodiments, the scope of which is defined in the claims and their equivalents. For example, while one use of the invention is in the medical field as a surgical scalpel, the same principles could be used to make devices for other medical and non-medical fields.

What is claimed is:

1. A utility tool device, comprising:
a handle with a front end, a rear end, a tool recess and a lever arm recess;
a rotatable lever arm with a magnet configured to fit in said lever arm recess; and
a rotatable lever hinge located closer to said rear end than said front end and attached to said lever arm and configured to attach to a tool;
wherein said tool recess is configured to have a tool fit in said tool recess;
wherein said magnet is configured to lock said tool in place when said lever arm is in said lever arm recess, such that said tool extends beyond said front end; and
wherein said rotatable lever hinge remains closer to said rear end than said front end when said tool is locked in place.

2. The utility tool device of claim 1, wherein said tool is a blade.

3. The utility tool device of claim 1, wherein said lever hinge is rotatable about an axis perpendicular to said tool and said lever arm is rotatable approximately 90 degrees about said lever hinge.

4. The utility tool device of claim 1, wherein said handle comprises a first handle portion and a second handle portion.

5. The utility tool device of claim 4, further comprising:
a second rotatable lever arm with a second magnet configured to fit in a second lever arm recess; and
a second rotatable lever hinge attached to said second lever arm and configured to attach to a tool;
wherein said second tool recess is configured to have a tool fit in said second tool recess; and
wherein said second magnet is configured to lock a tool in place when said second lever arm is in said second lever arm recess.
